(19) United States
(12) Patent Application Publication
(10) Pub. No.: US 2005/0065577 A1
(43) Pub. Date: Mar. 24, 2005

(54) Low level laser tissue treatment

(76) Inventors: Frank G. McArthur, Key West, FL (US); James C. Nairne, Dallas, TX (US)

Correspondence Address: WELSH & FLAXMAN LLC, 2450 CRYSTAL DRIVE, SUITE 112, ARLINGTON, VA, 22202, US

(21) Appl. No.: 10/667,894

(22) Filed: Sep. 23, 2003

(51) Int. Cl. A61N 1/00

(52) U.S. Cl. 607/88

(57) ABSTRACT

A method for the therapeutic treatment of biological tissue of a patient with a low level laser. The method is achieved by diagnosing the nature and extent of the tissue disorder, establishing at least one treatment area, exposing the treatment area to monochromatic, coherent light below the level necessary to cause thermal damage to the tissue being treated, wherein said light is in the near infrared portion of the electromagnetic spectrum, and treating the treatment area for sufficient treatment time to produce clinically beneficial effects by delivering a dosage greater than 20 joules/cm².

### BACKGROUND OF THE INVENTION

1. Field of the Invention

This invention relates to the treatment of biological tissue using low level laser therapy methods and devices.

2. Description of the Prior Art

Low Level Laser Therapy (LLLT) is a promising medical field, which has been rapidly evolving over the past thirty years. Doctors, scientists, and other researchers have discovered that the application of optical energy produced by lasers can have beneficial and healing effects when applied to living tissues. This type of therapy is called “low level” because the amount of laser energy applied to tissues is much lower than previous medical applications for lasers. For example, the laser levels used in LLLT are much lower than those used in cutting or ablating tissues during surgical procedures. In LLLT, the light produced by the laser is sufficiently low to avoid thermal damage to tissues. Instead, most of the optical energy reacts with the cells that make up the tissue, producing positive effects on cellular functions and healing processes. These positive effects are currently being used and investigated for treatment of a wide variety of musculoskeletal, soft tissue, and neurological conditions.

A recent and highly significant clinical trial on the therapeutic effects of low-intensity laser therapy shows support for “the idea that laser radiation at intensities too low to produce significant heating has beneficial therapeutic effects.” The results of this study found that “the group that received irradiation reported a consistent, time-dependent, statistically significant improvement compared with controls.” Jeffrey R. Basford, MD, Charles G. Sheffield, P T, and William S. Harmsen, B S, Laser Therapy: A Randomized, Controlled Trial of the Effects of Low Intensity Nd:YAG Laser Radiation on Musculoskeletal Back Pain, American Congress of Rehabilitation Medicine and the American Academy of Physical Medicine and Rehabilitation, 1999. While the full extent of the benefits of LLLT at the cellular level are still being discovered, several specific non-thermal effects are already known to have biostimulative properties that assist in the function and healing of living tissues. Optical energy stimulates microcirculation, assisting with cellular regenerative processes*. Jan Tuner and Lars Hode, Low Level Laser Therapy: Clinical Practice and Scientific Background, Prima Books AB ISBN 91-631-1344-9, 1999. One of the most experienced and respected researchers in the field of LLLT is responsible for investigation into other possible “primary mechanisms of light action on photoacceptor molecules.” Among the possible mechanisms discussed are “increasing superoxide production” and “the generation of singlet oxygen” within cells, both of which play key roles in the cellular respiratory function and production of ATP. Tiina Karu, The Science of Low Power Laser Therapy, Gordon and Breach Science Publishers, pp. 68-73, ISBN 90-5699-108-6.

A variety of investigations and studies into the effects of LLLT have been performed over the past three decades. The methods of treatment researched in theses studies generally differ from one another in two main areas: wavelength and dosage.

Laser light is produced by pumping energy into a lasing medium, such as a rod of semi-conducting material or a tube of certain gases (like CO₂). The energy that is subsequently emitted by the lasing medium is generally coherent and monochromatic in nature. Monochromatic means that the light coming from a laser is all of the same wavelength (as opposed to a regular light bulb, for example, which emits light at many different wavelengths). Coherent means that the waves of light have similar direction, amplitude, and phase to each other. Lasers are generally differentiated by the type of lasing material used and, therefore, by the wavelength they produce.

The wavelength of light produced by a laser affects the amount that the light is absorbed, reflected, or scattered when it encounters different materials, such as hemoglobin, melanin, and water (three primary substances found in human tissues). For some medical applications of LLLT, treatment of tissue near the skin is desired, and wavelengths with higher absorption are used. However, when the tissue to be treated is located deeper, less absorption, reflection, and scattering is desired. This property of the optical energy is known as penetration, and the tissue penetration for different wavelengths of light is shown in the graph and table presented as FIGS. 1 and 2.

Based on the data presented in FIG. 1 regarding the penetration of light through tissue, researchers have determined that optimal penetration occurs with light energy in the red to infrared range. Most prominently, AlGaAs light-emitting diodes (830 nm wavelength) and Nd:YAG lasers (1064 nm wavelength) have been used to achieve higher penetration of tissue for the treatment of deeper-lying medical conditions.

The second major area of research in LLLT is in the dosage. Dosage is measured in joules per square centimeter (J/cm²), and is determined by the energy output of the laser (measured in watts), the time the tissue is treated (seconds), and the size of the area treated by the laser (square centimeters). For example, a 1 watt laser applied to a 1 cm² area for 1 second would provide a dosage of 1 J/cm².

In the past, therapeutic use of low level lasers was limited to very low dosages of optical energy, generally restricted to 1 to 4 J/cm². Over time, slightly higher power lasers of 30 to 100 or more milliwatts have been investigated. Prior to the development of the invention disclosed and claimed in the present specification, the highest known dosage to be used in LLLT was 15 J/cm², and in that case the optical energy was being used to produce thermal effects rather than non-thermal ones. U.S. Pat. Nos. 5,445,146 and 5,951,596 to Beilinger.

Based on the data from research performed in the prior art, a clear goal of LLLT is to maximize the non-thermal, biostimulative effects of optical energy on tissues, while minimizing unwanted and potentially damaging thermal effects. By carefully controlling the power density of the optical energy as well as the treatment area and time, the present invention offers positive therapeutic effects, using higher dosages of biostimulating energy than has been achieved in the past, while avoiding unwanted thermal effects.

### SUMMARY OF THE INVENTION

It is, therefore, an object of the present invention to provide a method for the therapeutic treatment of biological tissue of a patient. The method is achieved by diagnosing the nature and extent of the tissue disorder, establishing at least one treatment area, exposing the treatment area to monochromatic, coherent light below the level necessary to cause thermal damage to the tissue being treated, wherein said light is in the near infrared portion of the electromagnetic spectrum, and treating the treatment area for sufficient treatment time to produce clinically beneficial effects by delivering a dosage greater than 20 joules/cm².

It is also an object of the present invention to provide a method for the therapeutic treatment of biological tissue wherein the monochromatic, coherent light has a wavelength between approximately 700 and approximately 1400 nm.

It is another object of the present invention to provide a method for the therapeutic treatment of biological tissue wherein an Nd:YAG laser source is used to produce monochromatic, coherent light of 1064 nm wavelength.

It is a further object of the present invention to provide a method for the therapeutic treatment of biological tissue wherein the treatment time is less than approximately 20 minutes per treatment area.

It is yet another object of the present invention to provide a method for the therapeutic treatment of biological tissue wherein the treatment time is approximately 90 seconds per treatment area is used.

It is still a further object of the present invention to provide a method for the therapeutic treatment of biological tissue wherein the treatment area is between 0.1 and 100 cm².

It is also another object of the present invention to provide a method for the therapeutic treatment of biological tissue wherein the treatment area is 10 cm².

It is another object of the present invention to provide a method for the therapeutic treatment of biological tissue wherein the dosage is between approximately 20 and 100 J/cm².

It is a further object of the present invention to provide a method for the therapeutic treatment of biological tissue wherein the dosage is approximately 45 J/cm² dosage.

It is also a further object of the present invention to provide a method for the therapeutic treatment of biological tissue wherein multiple treatment areas are treated.

It is still another object of the present invention to provide a method for the therapeutic treatment of biological tissue wherein treatment is repeated daily, or periodically for a prescribed number of days necessary to produce clinically beneficial effects.

It is also an object of the present invention to provide a method for the therapeutic treatment of biological tissue wherein the light is delivered in a continuous wave.

It is another object of the present invention to provide a method for the therapeutic treatment of biological tissue wherein the light is produced by a light source operated in a pulsing fashion.

It is also an object of the present invention to provide a method for the therapeutic treatment of biological tissue wherein the tissue disorder is acute due to trauma.

It is also an object of the present invention to provide a method for the therapeutic treatment of biological tissue wherein the tissue disorder is chronic.

It is also an object of the present invention to provide a method for the therapeutic treatment of biological tissue wherein the application of LLLT at the wavelengths and dosages specified results in musculoskeletal pain management.

It is a further object of the present invention to provide a method for the therapeutic treatment of biological tissue wherein the tissue disorder is selected from the group consisting of inflammatory arthritis, soft tissue wounds, osteoarthritis, sports injuries, tendinitis, neuropathic pain, nerve repair, oro-facial pain, acute and chronic musculoskeletal pain, hemangiomas, tinnitus, immune modulation, patellofemoral pain, bactericidal effects and Pyronie's disease.

Other objects and advantages of the present invention will become apparent from the following detailed description when viewed in conjunction with the accompanying drawings, which set forth certain embodiments of the invention.

### BRIEF DESCRIPTION OF THE DRAWINGS

FIG. 1 is a graph of the absorption coefficient for various biological components versus wavelength. FIG. 2 is chart showing tissue penetration as a function of wavelength.

### DESCRIPTION OF THE PREFERRED EMBODIMENTS

The detailed embodiments of the present invention are disclosed herein. It should be understood, however, that the disclosed embodiments are merely exemplary of the invention, which may be embodied in various forms. Therefore, the details disclosed herein are not to be interpreted as limiting, but merely as the basis for the claims and as a basis for teaching one skilled in the art how to make and/or use the invention.

The present invention maximizes the non-thermal, biostimulative effects of optical energy on tissues, while minimizing unwanted and potentially damaging thermal effects. By carefully controlling the power density of the optical energy as well as the treatment area and time, the present method offers positive therapeutic effects using relatively high dosages of biostimulating energy, while avoiding unwanted thermal effects.

The method is generally achieved in the following manner. The nature and extent of the tissue disorder is first diagnosed and the location, size, and number of treatment area(s) is established. Thereafter, the treatment area(s) is exposed to monochromatic, coherent light below the level necessary to cause thermal damage to the tissue being treated, wherein the light is in the near infrared portion of the electromagnetic spectrum. The treatment area is exposed to the monochromatic, coherent light for a sufficient treatment time to produce clinically beneficial effects by delivering a dosage greater than 20 joules/cm². As discussed above, monochromatic light refers to light which is all of the same wavelength and coherent means that the waves of light have similar direction, amplitude, and phase to each other. In accordance with a preferred embodiment of the present invention, monochromatic, coherent light having a wavelength in the 700 to 1400 nanometer (nm) range is used in the treatment of soft tissue disorders by irradiating a treatment area for sufficient time to produce clinically beneficial effects. This is achieved by delivering a dosage greater than 20 Joules per square centimeter (J/cm²). It is contemplated dosages in the range of approximately 20 J/cm² to approximately 100 J/cm² are preferred, although those skilled in the art might appreciate applications requiring high dosages without departing from the spirit of the present invention. In a preferred embodiment, a dosage of approximately 45 J/cm² is delivered to the tissue. Additional treatments and treatment areas maybe treated as determined by the diagnosing physician.

By way of example, a laser generating 5 Watts of output power is used to deliver optical energy to a 10 cm² treatment area. This produces a power density of 500 mW/cm². This power density is applied for 90 seconds to produce a dosage of 45 Joules/cm² at the treatment area. As those skilled in the art will certainly appreciate, dosage is the energy density delivered to the treatment area, as described in accordance with the following equation:
Energy Density (Joules/cm²)=Power Density (Watts/cm²)×Time (seconds).
Joules are the unit used to measure energy, and can also be called Watt-seconds. The equation for energy is:
Energy Joules)=Power (Watts)×Time (seconds).

The method in accordance with the present invention is adapted for the therapeutic treatment of any of a patient's muscle, nerve, epithelial, and/or connective tissues. In accordance with a preferred embodiment, the method is adapted for the relief of acute and chronic soft tissue trauma and to provide musculoskeletal pain management. Other applications include, but are not limited to, inflammatory arthritis, soft tissue wounds, osteoarthritis, sports injuries, , tendinitis, neuropathic pain, nerve repair, oro-facial pain, acute and chronic musculoskeletal pain, hemangiomas, tinnitus, immune modulation, patellofemoral pain, bactericidal effects, and Pyronie's disease.

In accordance with a preferred embodiment of the present invention, the tissue to be treated is irradiating with optical energy. The treatment with optical energy increases microcirculation and creates biostimulative effects at the cellular level. Photobiostimulation occurs when monochromatic, coherent light acts on the photoacceptor molecules within the cells of the tissue being treated. Following photoexcitation of the electronic states of carriers, changes in their redox properties cause electron transfer to be accelerated on the respiratory chain. In mitochondrial electron transport the superoxide radical is produced. Photoexcitation also increases superoxide production, which is thought to be a source of electrons for the oxydative phosphorylation of ADP, resulting in increased ATP production. By altering the cellular redox state, significant clinically beneficial results are achieved.

More particularly, the applied optical energy is the form of monochromatic, coherent light having wavelength between approximately 700 and approximately 1400 nanometers (nm). In accordance with a preferred embodiment, a diode-pumped, solid-state Nd:YAG laser is used as the source of this optical energy, producing light at 1064 nm wavelength. This light is guided through a fiber optic cable to a handpiece with an aperture of a size appropriate for the desired treatment area. The size of the aperture is chosen based upon the size of the treatment area and the time necessary to cover the treatment area. Laser systems such as those disclosed in U.S. Pat. No. 5,521,936 to Irwin, entitled “Radial Laser Diode Array”, and U.S. Pat. No. 5,627,850 to Irwin et al., entitled “Laser Diode Array”, which are incorporated herein by reference may be utilized in applying desired dosages in accordance with the present invention. While preferred laser systems are contemplated for use in accordance with the present invention, those skilled in the art will appreciate the variety of laser systems which may be utilized without departing from the spirit of the present invention.

In accordance with one embodiment, the aperture of the handpiece is circular, with an area of 10 cm². By providing an aperture of 10 cm², this embodiment is particularly adapted for application to a treatment sight of approximately 10 cm².

By way of example, the present method is disclosed. After diagnosing the nature and extent of a tissue disorder, the physician establishes the location, size, and number of treatment areas. In one embodiment, the size of the treatment areas is approximately 10 cm², although the treatment area may range from between approximately 0.1 cm² to approximately 100 cm². Where different treatment sights are contemplated, the laser aperture is accordingly adjusted to accommodate the different treatment sight sizes; for example, an aperture of 0.1 cm² is utilized for a treatment sight of 0.1 cm² and an aperture of 100 cm² is utilized for a treatment sight of 100 cm². or alternatively, the 100 cm² treatment area may be treated by moving the 10 cm² aperture over the entire area in a grid-like fashion. The physician then exposes the treatment area to monochromatic, coherent light delivered from the handpiece as described above.

However, those skilled in the art will appreciate the various ways in which the desired dosage maybe achieved. Considering the formula for calculating dosages as discussed above, that is, Dosage=Power Density×Time, treatment times can vary considerably depending on the power density being used. For example, a dosage of 45 J/cm² may be achieved in a variety of ways. In accordance with a preferred method, 5 Watt laser, a 10 cm² treatment area (i.e. 500 mW/cm² power density), and a 90 second treatment time are used. Another method would be to use a 1 Watt laser, which would produce only 100 mW/cm² power density, and would, therefore, require 450 seconds to produce the desired dosage of 45 J/cm². Conversely, a power density of 1000 mW/cm² could be used (10 W laser, 10 cm² spot size), requiring only 45 seconds to achieve the same 45 J/cm dosage.

While the exact power density required to damage tissue varies from person to person, and by wavelength, there is a reasonable limit to how high a power density should ever be used, and, therefore, there is a lower limit to the duration of the treatment time which may be used. 2,000 mW/cm² is a very high power density and would likely cause burning of tissue. If this power density were used in the example above (10 cm² spot size), the treatment time would be shortened to 22.5 seconds in order to produce 45 J/cm². However, all this calculation is based upon the assumption a continuous wave (CW) laser is being used. If pulsing (or “Q-switching”) is used, the treatment times can be significantly shorter. This is because pulsed lasers provide higher power density “bursts” of optical energy, potentially allowing higher dosages to be administered in less time. For this reason, it is contemplated the minimum treatment time duration is between 1 to 10 seconds.

It is contemplated the maximum duration for treatment time is approximately less than 20 minutes. This limit has been established based upon the understanding that it is unlikely a power density of 100 mW/cm² will be used and the upper dosage as defined is 100 J/cm². This would dictate a maximum treatment time of 1,000 seconds or 16.7 minutes (100 J/cm²=100 mW/cm²×1000 seconds). A 20 minute maximum allows one to dip below the 100 mW/cm² power density mark a little if they wish.

It will be appreciated by those skilled in the art that the treatment may be a single treatment or may be repeated daily, or periodically for a prescribed number of days necessary to produce clinically beneficial effects. In addition, the light maybe provided in a continuous wave or in a pulsing manner.

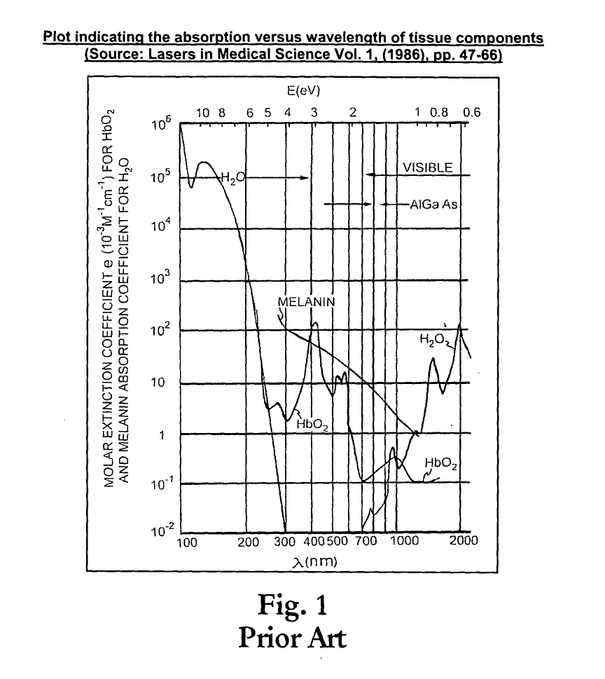

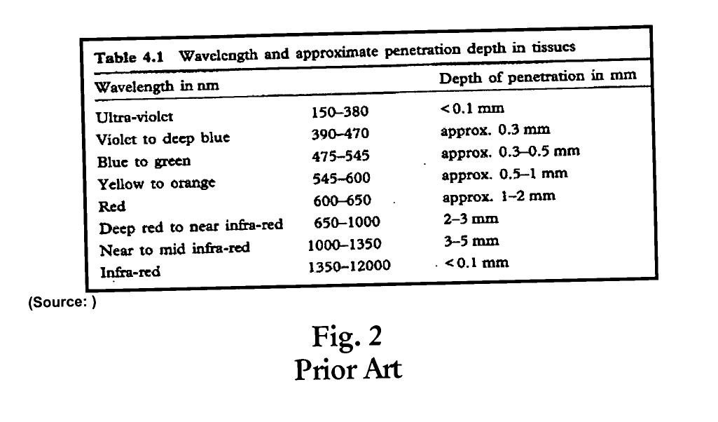

FIG. 2 illustrates the relationship between various wavelengths of light and the depth of penetration into the patient's tissues. The choice of wavelength used is dependent upon the depth required to treat the affected tissues. The patient's level of melanin must also be considered. As illustrated in FIG. 1, the higher the wavelength the less absorption by melanin.

Although the application of low levels of laser light to produce a therapeutic effect has been used in Europe for more than twenty years, the combination of very low power densities and small treatment area sizes used in the prior art resulted in very low dosages being delivered. In fact, most protocols call for dosages of less than 10 J/cm². In addition, most of the lasers used heretofore have produced light of shorter wavelengths, many in the visible red portion of the spectrum. The present invention uses longer wavelength infrared light for greater tissue penetration, and the protocol dictates much higher dosages of photon energy. This results in a dramatic therapeutic effect, generally accompanied by immediate pain relief.

While the preferred embodiments have been shown and described, it will be understood that there is no intent to limit the invention by such disclosure, but rather, is intended to cover all modifications and alternate constructions falling within the spirit and scope of the invention as defined in the appended claims.

1. A method for the therapeutic treatment of biological tissue of a patient, comprising the following steps:
diagnosing the nature and extent of a tissue disorder;
establishing the location and size of at least one treatment area;
exposing the at least one treatment area to monochromatic, coherent light below the level necessary to cause thermal damage to the tissue being treated, wherein the light is in the near infrared portion of the electromagnetic spectrum; and
treating said treatment area for sufficient treatment time to produce clinically beneficial effects by delivering a dosage greater than 20 joules/cm².

2. The method according to claim 1, wherein the monochromatic, coherent light has a wavelength between approximately 700 and approximately 1400 nm.

3. The method according to claim 2, wherein an Nd:YAG laser source is used to produce monochromatic, coherent light of 1064 nm wavelength.

4. The method according to claim 1, wherein the treatment time is less than approximately 20 minutes per treatment area.

5. The method according to claim 4, wherein the treatment time is approximately 90 seconds per treatment area is used.

6. The method according to claim 1, wherein the treatment area is between 0.1 and 100 cm².

7. The method according to claim 6, wherein the treatment area is 10 cm².

8. The method according to claim 1, wherein the dosage is between approximately 20 and 100 J/cm².

9. The method according to claim 8, wherein the dosage is approximately 45 J/cm² dosage.

10. The method according to claim 1, wherein multiple treatment areas are treated.

11. The method according to claim 1, wherein treatment is repeated daily, or periodically for a prescribed number of days necessary to produce clinically beneficial effects.

12. The method according to claim 1, wherein the light is delivered in a continuous wave.

13. The method according to claim 1, wherein the light is produced by a light source operated in a pulsing fashion.

14. The method according to claim 1, wherein the tissue disorder results from acute trauma.

15. The method according to claim 1, wherein the tissue disorder is selected from the group consisting of inflammatory arthritis, soft tissue wounds, osteoarthritis, sports injuries, tendinitis, neuropathic pain, nerve repair, oro-facial pain, acute and chronic musculoskeletal pain, hemangiomas, tinnitus, immune modulation, patellofemoral pain, bactericidal effects and Pyronie's disease.
